(19) United States
(12) Patent Application Publication
(10) Pub. No.: US 2024/0331878 A1
(43) Pub. Date: Oct. 3, 2024

(54) PATIENT PRIVACY COMPLIANT TARGETING SYSTEM AND METHOD

(71) Applicant: IQVIA Inc., Durham, NC (US)

(72) Inventors: Yong CAI, Marina, CA (US); Yanping LIU, Harleysville, PA (US); Ruoxin LI, Chapel Hill, NC (US); Emily ZHAO, Wayne, PA (US); Yilian YUAN, North Wales, PA (US)

(21) Appl. No.: 18/193,918

(22) Filed: Mar. 31, 2023

(51) Int. Cl. G16H 50/70; G06N 3/08

(52) U.S. Cl. CPC G16H 50/70; G06N 3/08

(57) ABSTRACT

A method includes receiving data and integrating the data into a computing system. The method also includes applying a machine learning system to identify patients from the integrated data to place in one or more communities that include consumer-related data and social determinants of health data. The method also includes combining path projection, aggregation, and embedding to establish one or more paths to connect the patients to the communities based on the consumer-related data and/or the social determinants of health data in the one or more communities. The method also includes training a machine learning system to identify a correct path among the one or more established paths to place the patients on to be connected to the one or more communities.

### TECHNICAL FIELD

The present disclosure generally relates to identifying communities to place patients based on consumer data, neighbor data and social determinants of health data within the communities.

### BACKGROUND

Classic machine learning systems can attempt to connect patients to communities that best serve the needs of patients. The machine learning systems can take the individual information of the patients. The individual information of the patients can reveal the patients medical and economic situation. The individual information of the patients can also include what the patients may need the most to survive or get through a current predicament. However, the patients individual information may lack sufficient context with how each patient will survive within one or more communities.

Another drawback is the use of classic machine learning systems. The classic machine learning systems may be limited in identifying the optimal communities to place patients. The classic machine learning systems may just be using past information of the patients without identifying which communities more closely resemble the needs of the patients.

Current machine learning systems may identify consumer attributes of the patients. However, the current machine learning systems may not be able to identify which communities share similar consumer attributes with the patients. Other connections such as neighbor or community relations, demographic factors are not applied to identify communities for patients and individuals. As such, the machine learning systems may identify communities that are not ideal for the patients based on the consumer attributes of the patients.

Accordingly, a need exists for a machine learning system that can more accurately predict communities to place patients within based on the needs of the patients. Moreover, a need exits to identify more pertinent factors for the different types of patients. Further, based on identifying the various needs of the different patients, the machine learning system should more accurately place the patients in the communities that best serve their needs.

### SUMMARY

The following summary is provided to facilitate an understanding of some of the features of the disclosed embodiments and is not intended to be a full description. A full appreciation of the various aspects of the embodiments disclosed herein can be gained by taking the specification, claims, drawings, and abstract as a whole.

The aforementioned aspects and other objectives can now be achieved as described herein.

In an embodiment, a computer-implemented method comprises receiving data from a plurality of databases. The data is integrated into a computing system. The method also includes applying a machine learning system to identify patients from the integrated data to place in one or more communities. The one or more communities include consumer-related data and social determinants of health data. The method also includes combining path projection, aggregation, and embedding to establish one or more paths to connect the patients to the one or more communities based on the consumer-related data and/or the social determinants of health data in the one or more communities. The method also includes training the machine learning system to identify a correct path among the one or more established paths to place the patients on to be connected to the one or more communities.

The method also includes creating one or more algorithms to project the patients into the one or more communities that include the social determinants of health data.

The method also includes identifying information among physician network communities within the integrated data to further identify the one or more communities in which to place the patients.

In an embodiment, a computer program product is configured to receive data from a plurality of databases. The computer program product also applies a machine learning system to identify patients from the integrated data to place in one or more communities. The one or more communities include consumer-related data and/or social determinants of health data. The computer program product also combines path projection, aggregation, and embedding to establish one or more paths to connect the patients to the communities based on the consumer-related data and the social determinants of health data in the one or more communities. The computer program product also trains the machine learning system to identify a correct path among the one or more established paths to place the patients on to be connected to the one or more communities.

Data from physician networks within the one or more communities is collected to further identify the one or more communities with which to place at least one or more of the patients.

One or more algorithms are used to place the patients in the one or more communities that include the social determinants of health data.

A second noise function is applied when patient and personal and/or confidential information within the blocks of data contain the one or more violations.

In an embodiment, a system includes one or more processors. The one or more processors collect one or more datasets of information. The one or more processors also separate the one or more datasets into respective blocks of data. Further, the one or more processors determine whether the information within the blocks of data are consistent or if one or more violations appear within the blocks of data. The one or more processors also apply a first noise function based on the determination that the information within the blocks of data are consistent. The first noise function is applied when a loss off privacy and/or confidentiality exceeds a threshold. The one or more processors also display the blocks of data with the first noise function.

The blocks of data are displayed with the first noise function combined with a second noise function when the one or more violations occur.

The first noise function is combined with a second noise function to remove or correct the one or more violations within the blocks of data.

### BRIEF DESCRIPTION OF THE FIGURES

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention.

FIG. 1 illustrates a computing system in accordance with an embodiment of the invention;

FIG. 2 illustrates a geo-spaitial coded heterogenous graph network in accordance with an embodiment of the invention;

FIG. 3 illustrates a geo-encoded heterogenous graph neural network architect in accordance with an embodiment of the invention;

FIG. 4 illustrates an application workflow in accordance with an embodiment of the invention; and

FIG. 5 illustrates a flow chart in accordance with an embodiment of the invention.

Unless otherwise indicated illustrations in the figures are not necessarily drawn to scale.

### DETAILED DESCRIPTION OF SOME EMBODIMENTS

### Background and Context

Subject matter will now be described more fully herein after with reference to the accompanying drawings, which form a part hereof, and which show, by way of illustration, specific example embodiments. Subject matter may, however, be embodied in a variety of different form and, therefore, covered or claimed subject matter is intended to be construed as not being limited to any example embodiments set forth herein, example embodiments are provided merely to be illustrative. Likewise, a reasonably broad scope for claimed or covered subject matter is intended. Among other issues, subject matter may be embodied as methods, devices, components, or systems. The followed detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” and variations thereof as utilized herein may not necessarily refer to the same embodiment and the phrase “in another embodiment” or “in another example embodiment” and variations thereof as utilized herein may or may not necessarily refer to a different embodiment. It is intended, for example, that claimed subject matter include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Generally, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as a “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

One having ordinary skill in the relevant art will readily recognize the subject matter disclosed herein can be practiced without one or more of the specific details or with other methods. In other instances, well-known structures or operations are not shown in detail to avoid obscuring certain aspects. This disclosure is not limited by the illustrated ordering of acts or events, as some acts may occur in different orders and/or concurrently with other acts or events. Furthermore, not all illustrated acts or events are required to implement a methodology in accordance with the embodiments disclosed herein.

Unless defined otherwise, all technical and scientific terms used herein have the same meanings as commonly understood by one of ordinary skill in the art to which the disclosed embodiments belong. Preferred methods, techniques, devices, and materials are described, although any methods, techniques, devices, or materials similar or equivalent to those described herein may be used in the practice or testing of the present invention.

Although claims have been included in this application to specific enumerated combinations of features, it should be understood the scope of the present disclosure also includes any novel feature or any novel combination of features disclosed herein.

References “an embodiment,” “example embodiment,” “various embodiments,” “some embodiments,” etc., may indicate that the embodiment(s) so described may include a particular feature, structure, or characteristic, but not every possible embodiment necessarily includes that particular feature, structure, or characteristic.

Headings provided are for convenience and are not to be taken as limiting the present disclosure in any way.

Each term utilized herein is to be given its broadest interpretation given the context in which that term is utilized.

### Terminology

The following paragraphs provide context for terms found in the present disclosure (including the claims):

The transitional term “comprising”, which is synonymous with “including,” “containing,” or “characterized by,” is inclusive or open-ended and does not exclude additional, unrecited elements or method steps. See, e.g., Mars Inc. v. H.J. Heinz Co., 377 F.3d 1369, 1376, 71 USPQ2d 1837, 1843 (Fed. Cir. 2004) (“[L]ike the term ‘comprising,’ the terms ‘containing’ and ‘mixture’ are open-ended.”). “Configured to” or “operable for” is used to connote structure by indicating that the mechanisms/units/components include structure that performs the task or tasks during operation. “Configured to” may include adapting a manufacturing process to fabricate components that are adapted to implement or perform one or more tasks.

“Based On.” As used herein, this term is used to describe factors that affect a determination without otherwise precluding other or additional factors that may affect that determination. More particularly, such a determination may be solely “based on” those factors or based, at least in part, on those factors.

All terms of example language (e.g., including, without limitation, “such as”, “like”, “for example”, “for instance”, “similar to”, etc.) are not exclusive of other examples and therefore mean “by way of example, and not limitation . . . ”.

A description of an embodiment having components in communication with each other does not infer that all enumerated components are needed.

A commercial implementation in accordance with the scope and spirit of the present disclosure may be configured according to the needs of the particular application, whereby any function of the teachings related to any described embodiment of the present invention may be suitably changed by those skilled in the art.

The flowchart and block diagrams in the figures illustrate the architecture, functionality, and operation of possible implementations of systems and methods according to various embodiments. Functions noted in the block may occur out of the order noted in the figures. For example, two blocks shown in succession may, in fact, be executed substantially concurrently, or the blocks may sometimes be executed in the reverse order, depending upon the functionality involved.

Further, any sequence of steps that may be described does not necessarily indicate a condition that the steps be performed in that order. Some steps may be performed simultaneously.

### INTRODUCTION

Embodiments of the present invention include a machine learning system that enables patients represented by patient data to be placed in one or more communities that will best serve the patients based on the needs of the patients. The computing system will be able to identify with greater targeting accuracy the communities that would best serve the needs of the patients represented by the patient data. A computing system that incorporates the machine learning system will receive patient data from one or more databases.

The patient data will include consumer data, neighbor data, and social determinants of health data. The consumer data will indicate the consumer behaviour of patients with respect to buying, shopping, and purchasing interests of the patients. The community/neighbor data will how patients represented by the community data are involved in their various community and how they interact with their neighbors. Moreover, the community data can include how the patients participate in within the various communities that the patients have been a part of. The community/neighbor data will include data on patients that illustrates who the patients interact within the communities, and how the patients interact with their friends and neighbors within the communities. Information among physician network communities can be identified with respect to the neighbor/community data and also with the consumer data mentioned above. Moreover, data from the physician networks can be collected from the consumer data and neighbor data. Patients placed within the consumer and neighbor communities will be able to interact with the healthcare facilities within the communities. The social determinants of health data can include the age, ethnicity, occupation, social status, gender, and income of the patients within the communities. The various communities can include health centers, hospitals, community centers, shopping centers, universities, and places of business and physician network communities. Data from the physician networks can also be collected for the social determinants of health data as well.

Meta-path projection, neighbor aggregation, and feature embedding can be used to identify the paths to the respective communities. The meta-path projection, neighbor aggregation, and feature embedding can be combined to identify the paths to connect the communities based on the community data, neighbor data, and social determinants of health data. The path or paths will enable the patients to be connected to the communities based on their data and on their needs.

Once the patient data is received within the computing system, the patient data is used as training data to train the machine learning system. The machine learning system is trained to predict the path that the patients should be placed on to connect to their communities. The patients represented by the consumer data should be placed on the path to connect to the communities based on consumer data. Similarly, the patients represented by the community/neighbor data and social determinants of health data should be placed on the path or paths to connect to the communities based on community/neighbor data and social determinants of health data respectively.

The trained machine learning system can be applied to produce outputs that include a patient scoring model and geo-embedding coding and data based on the consumer data, neighbor data, and social determinants of health data. The privacy of the outputs will be protected as the trained machine learning system produces the outputs. The patient scoring model and geo-embedding data can be used to identify and place the patients on the right paths to be connected to the right communities. As such, based on the patient scoring model and geo-embedding data, the patients represented by the community data will be placed on the path to connect to one or more communities that meets their needs with greater accuracy than prior systems.

The communities based on consumer data can include research centers, shopping centers, retail areas, and places of business. Referral networks can also be within the consumer communities. The communities based on neighbor/community data will include friends and neighbors of the patients and individuals that the patients would engage with and prefer to interact with. The neighbor/community data can also include patients with similar attributes and geographic topology. Community centers and places of worship can also be within the neighbor communities.

The communities based on the social determinants of health data will include people with similar social, ethnic, demographic backgrounds, and economic status that are similar to the patients represented by the social determinants of health data. Jobs and other opportunities that fit the patients needs based on the social determinants of health data will also be within the communities based on social determinants of health data. Environmental factors and sociometric ties can also be within the social determinants of health data. Businesses and schools that meet the demographic, economic and social needs of the patients will be within such communities.

Accordingly, the patients will be more accurately connected to the communities that best meet their needs. The system will creating one or more algorithms to project the patients into the communities that include the consumer data, neighbor/community data, and social determinants of health data.

### System Structure

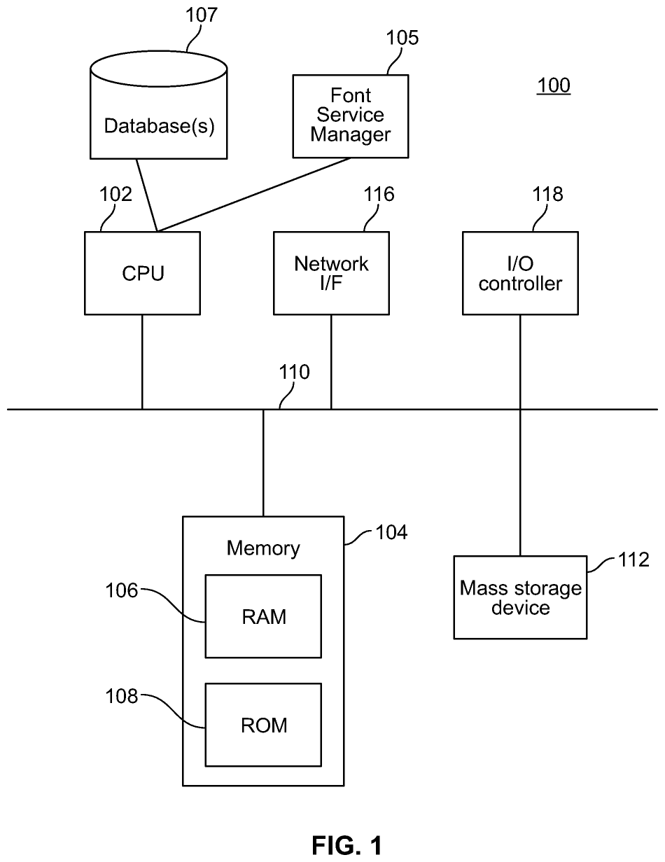

FIG. 1 illustrates a computing system 100. The computing system 100 includes a computer-processing unit (CPU) 102. The CPU 102 is connected to memory 104 with a random access memory (RAM) 106 and read only memory (ROM) 108. The CPU 102 is also connected to one or more databases 107 and a fond service manager 105. A communication interface 110 can enable the CPU 102 to be connected to a network 116 and a controller 118. A mass storage device 112 is also coupled to the controller 118.

In FIG. 1, in an embodiment, the CPU 102 can collect one or more datasets of information from the one or more databases 107. The datasets of information can include information on patients. The information on patients will include consumer data, neighbor data, and social determinants of health data. The consumer data will include the consumer-related behavior of the patients. The neighbor data can also include the community-related activities of the patients. The community data can also include, but is not limited to, the interests of the patients within the communities, and the behavior of the patients in relation to education, consumer spending, and personal interests of the patients. The neighbor data will include the interactions that the patients tend to have with various individuals within a community. The social determinants of health data will include, but are not limited to, social and economic status of patients, environmental information, employment information, sex, ethnicity, and sociometric ties to the communities. As such, the CPU 102 will employ an artificial intelligence machine learning system with one or more algorithms to use the consumer data, neighbor data, and social determinants of health data to more specifically identify what communities to place the patients within.

Referring to FIG. 1, a system will be applied that includes geo-spatial coded heterogenous graph networks on local and collaborative communities. Within the system, the consumer data and neighbor data will have layers for consumers and neighbor data. In addition, the social determinants of health data will also have a layer. Meta-path projection, neighbor aggregation, and feature embedding will be used on the consumer, neighbor, and social determinants of health layers. As a result of the meta-path projection, neighbor aggregation, and feature embedding, paths to the communities will be identified. The machine learning system/model will be trained with the input patient data to predict the path of the path of the patients. The trained machine learning system will be applied to produce outputs to place the patients onto the communities based on the consumer data, neighbor data, and/or the social determinants of health data. The system will be designed to more accurately provide the right path to place the patients on to connect the patients to the proper communities based on consumer data, neighbor data, and/or social determinants of health data.

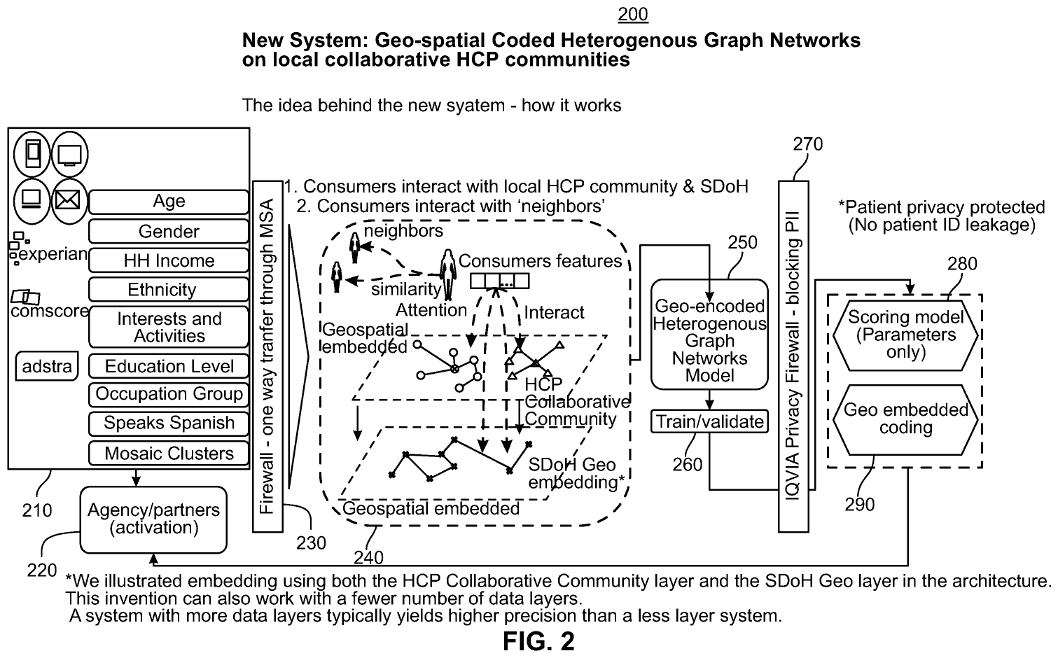

Referring to FIG. 2, a system 200 is illustrated that will enable a machine learning system or model to predict the path to place the one or more patients on. The predicted path will enable the patients to be connected to the communities based on consumer data, neighbor data, and/or social determinants of health data. The system 200 will train a machine learning system to predict the path that the patients represented by received patient data should be placed on to be connected to various communities.

In FIG. 2, one or more databases 210 are shown that include patient data. The patient data will be training data that will be input into a machine learning system to train the machine learning system to predict the path for each of the patients. The patient data will include information age, gender, income, and ethnicity. Such factors will typically be part of social determinants of health data. Interests and activities are also shown. Moreover, interests and activities can be part of consumer behavior and neighbor behavior that can be found within the community data and neighbor data. Education level and occupation group will typically be found with social determinants of health data within the communities. The Spanish speaking and mosaic clusters can be found in neighbor and consumer behavior within the communities. Patients who speak a common language can interact with each other within the same cluster in the neighbor/community data. Agency/partners activation 220 is also shown. The agency/partners activation 220 will connect the patient data to the machine learning system that will be used to predict the path to place the patients on to connect the patients to the communities.

Referring to FIG. 2, the received patient data consisting of consumer data, neighbor data, and social determinants of health data are passed through the firewall 230. After the patient data is passed through the firewall 230, the patient data goes to a stage 240 where the patient data undergoes meta-path projection, neighbor aggregation, and feature embedding. The consumer data, neighbor data, and social determinants of health data each undergo meta-path projection, neighbor aggregation, and feature embedding within the stage 240. The purpose of the meta-path projection, neighbor aggregation, and feature embedding is to identify the paths for the patients represented by the patient data to be placed on to be connected to the one or more communities. The machine learning system will be trained using the patient data from to predict the path to place the patients, represented by the patient data on. The path will connect the patients to the appropriate communities.

In FIG. 2, after the patient data undergoes the meta-path projection, neighbor aggregation, and feature embedding within the stage 240, updated patient data is passed onto a geo-encoded heterogenous graph networks model 250. The geo-encoded heterogenous graph networks model is a machine learning model or system. From the geo-encoded heterogenous graph networks model, the updated patient data is then passed onto stage 260 in which the machine learning system is trained or validated using the patient data to predict the path for the patients to connect to the various communities. After the machine learning system is trained/validated using the patient data, the patient data passes through a privacy firewall 270. As the updated patient data passes through the privacy firewall 270, the patient data is de-identified to protect patient privacy. The consumer data, neighbor data, and social determinants of health data are de-identified to protect patient privacy.

Referring to FIG. 2, after the updated patient data is passed through the privacy firewall 270, the trained machine learning system is applied to produce outputs. The outputs include a patient scoring model 280 with parameters only. With the scoring model 280, the updated patient data is scored to more closely identify what path each of the patients represented by the patient data should be placed on. The applying of the trained machine learning system also includes geo-embedded coding 290. The geo-embedded coding 290 is used on the consumer data, neighbor data and social determinants of health data to also more closely identify the path or paths for the patients on to be placed on to connect with the right communities. The application of the trained machine learning system produces outputs that include a patient scoring model and geo-embedded coding to more accurately connect the patients to the appropriate communities that more accurately meet the their needs.

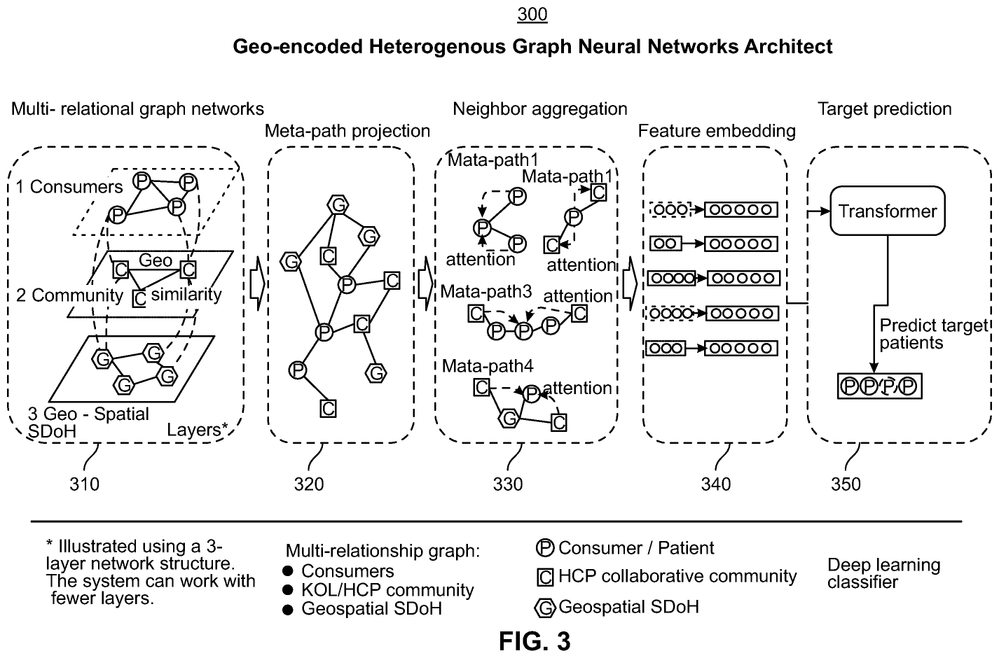

In FIG. 3, the geo-encoded heterogenous neural networks architect system 300 includes a machine learning system that is shown in further detail. Layers 310 of received patient data are illustrated that can be received from one or more databases. The geo-spacial layers 310 will include consumer data, neighbor/community data, and social determinants of health data. The consumer data will include the consumer behaviors of the patients within the communities. The consumer behavior can include what the patients like to buy or sell within the communities, and where the patients like to travel, and other interests of the patients. The neighbor data can include how the patients interact with their neighbors or other patients similar to them within communities. The social determinants of health data will include the ethnicity, education level, occupation, and income bracket of the patients.

Referring to FIG. 3, the received patient data will go through a meta-path projection 320. The meta-path projection 320 will more closely identify the path that the patients represented by the consumer data, community/neighbor data, and social determinants of health data. Moreover, the consumer data, community/neighbor data, and social determinants of health data can be linked together within the meta-path projection 320. The received patient data will also undergo neighbor aggregation 330. With neighbor aggregation 330, the meta-path 1 will illustrate attention to the consumer data. The meta-path 2 will show attention to the consumer data and community/neighbor data. The meta-path 3 will illustrate attention to the consumer data and community/neighbor data. The meta-path 4 will show attention to the consumer data, community/neighbor data, and social determinants of health data. The patient data will also undergo feature embedding 340 as well.

In FIG. 3, after the patient data has undergone meta-path projection 320, neighbor aggregation 330, and feature embedding 340, the paths to the communities have been identified. The paths will connect each of the patients represented by the patient data to the appropriate communities that best serve the patients needs. The consumer communities can include shopping and business centers. Hospital and research centers can also be within the consumer communities. The community/neighbor communities can include community centers, places of worship, and recreational centers such as gyms where neighbors can interact. The community/neighbor communities can also include hospitals as well. The social determinants of health communities can include ethnic centers, law firms, hospitals, universities, and places of business.

In FIG. 3, after the paths are identified, the machine learning system 300 can be trained with the patient data with target prediction 350. The updated patient data can be a training set of data that can be used to train the machine-learning system. The machine learning system 300 can be trained to accurately predict the path that the patients represented by the patient data should be placed on to be connected to the communities that represent consumer data, community/neighbor data, and/or social determinants of health data. The patients represented by the consumer data will be placed on a path that connects them to one or more of the communities that involve consumer data. Similarly, the patients represented by the neighbor data and social determinants of health data are also placed on a path to connect to the communities that include the neighbor data and social determinants of health data respectively.

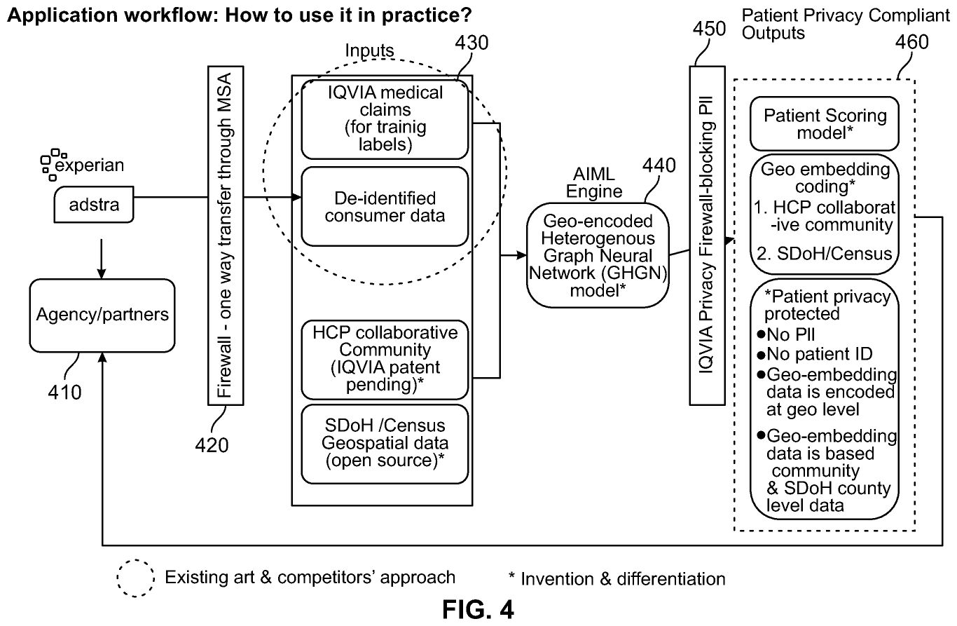

In FIG. 4, a system 400 is illustrated that shows an application workflow in which patients are connected to one or more communities based consumer data, neighbor data, and social determinants of health data. Consumer data, neighbor data, and social determinants of health data are passed through a firewall, and gathered as inputs. The inputs are then passed through a machine learning system. The machine learning system is trained to predict the path that the patients represented by the data should be placed on to be connected to the communities. After the machine learning system is trained, the trained machine learning system is applied to generate a patient scoring model and geo-encoded data.

Referring again to FIG. 4, patient data is fed from databases 405 into a fire wall 420. Agency/partners 410 also receive the patient data from the databases 405. The patient data passes through the firewall 420 and is organized as inputs 430. The inputs 430 include medical claims for training labels and de-identified consumer data. The inputs 430 will also include neighbor data or collaborative community data. Further, the inputs 430 will also include social determinants of health data. The consumer data, neighbor data, and social determinants of health data will each represent a plurality of patients.

In FIG. 4, the inputs 430 will be training data that is passed into a machine learning system 440. The machine learning system 440 will be trained using the inputs 430 that include the consumer data, neighbor data, and social determinants of health data. The training data or inputs 430 are then passed through a privacy firewall 450. The privacy firewall 450 will ensure that protected health information (PHI) data of the patients that are represented by the inputs 430 are protected. The trained machine learning system 440 is used to generate outputs 460. The outputs 460 include a patient scoring model. The patient scoring model will provide scores for the inputs 430 that will enable the patients represented by the inputs 430 to be connected to the appropriate communities. The outputs 460 will also include geo embedding coding for the consumer data, neighbor data, and social determinants of health data. Further the outputs 460 will protect the PHI for the patients that are represented by the consumer data, neighbor data, and social determinants of health data. The outputs 460 will provide patient scoring and geo-embedding coding of the consumer data, neighbor data, and social determinants of health data. As as result of the outputs 460, the patients that are represented by the community data, neighbor data, and social determinants of health data are placed on a path to be connect to the appropriate communities.

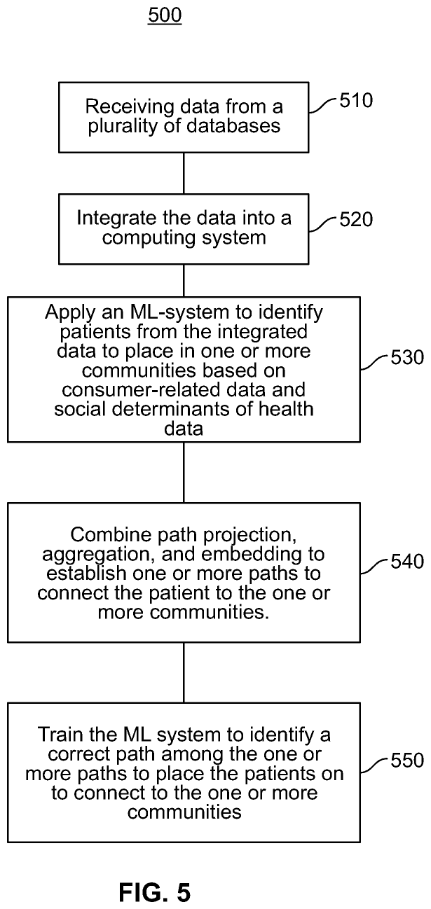

Referring to FIG. 5, a method 500 is illustrated that shows how a machine learning system receives an input of patient data from one or more databases. The patient data will include consumer data, neighbor data, and social determinants of health data. A plurality patients will be represented by the consumer data, neighbor data, and social determinants of health data. The patient data will be input data or training data that is put through a machine learning system. The machine learning system is trained with the input data. The trained machine learning system is then used to predict or identify a path that the patients should be placed on to connect the patients to the appropriate communities. The patients represented by the consumer data will be placed on a path that will connect them to communities that will better serve the patients represented by the consumer data. Similarly, the patients represented by the community and social determinants of health will be placed on a path to connect to the communities that are represented by the community data and social determinants of health data.

In FIG. 5, at step 510, data is received from one or more databases. As mentioned above, the data will be patient data based on consumer data, neighbor data, and social determinants of health data.

Referring to FIG. 5, at step 520, the input data consisting of the consumer data, neighbor/community data, and social determinants of health data is training data that is passed through a machine learning system. The machine learning system will ultimately be trained with the input data to identify patients from the integrated data to place in one or more communities based on the consumer data, neighbor data, and social determinants of health data.

In FIG. 5, at step 530, the machine learning system is used to identify patients from the patient data to eventually place in the one or more communities. The communities will include other patients, patient centers, meeting areas, places of business, hospitals, universities. As such, the machine learning system will receive the input of patient data and eventually be trained using the patient data to predict the path that the patients should be placed on to connect the various communities.

Referring to FIG. 5, at step 540, path projection, aggregation, and embedding is used to establish one or more paths to connect the patients represented by the patient data to the one or more communities. The established paths will be used to connect the patients to the communities after the machine learning system is trained with the input patient data.

In FIG. 5, at step 550, the machine learning system is trained with the input patient data. The machine learning system is trained with the training data (patient data) to identify predict the path among the one or more established paths to place the patients on to connect the patients to the one or more communities. After the machine learning system is trained with the input data, the trained machine learning system is applied to produce outputs that provide patient scoring and geo-embedding coding for the patients. Ultimately, the patients are connected on the paths to connect to the communities based on the outputs from the machine learning system.

Those skilled in the art will appreciate that the example embodiments are non-exhaustive and that embodiments other than that described here may be included without departing from the scope and spirit of the presently disclosed embodiments.

### Advantages/Summary

Overall, patient data will be retrieved from one or more databases into a computing system. The patient data will include consumer data, neighbor/community data, and social determinants of health data. The computing system will use the received patient data as training data to train a machine learning system. The machine learning system will be trained to predict and identify the communities to place the patients, represented by the patient data, into one or more communities. Patients represented by either the consumer data, community data, and/or social determinants of health data will be placed on a path to connect the communities that more closely represent their type of patient data.

Further, the path to the communities can be obtained by using a combination of meta-path projection, neighbor aggregation, and feature embedding. The combination of the meta-path projection, neighbor aggregation, and feature embedding will be used to obtain the paths to the communities. The patients that are represented by either the consumer data, neighbor data, and social determinants of health data will ultimately be placed on the paths to connect to the communities that also include the type of data which represent the patients.

As the path is created, and after the machine learning system is trained with the received patient data to identify the correct path to place the patients on, the trained machine learning system is applied to produce various outputs. The various outputs will include a patient scoring model based on the community data, neighbor data, and social determinants of health data. The outputs will include geo-embedded coding on the consumer data, neighbor data, and social determinants of health data. The privacy of the patients represented by consumer data, neighbor data, and social determinants of health data are protected when the trained machine learning system produces the outputs. As a result of the patient scoring models and geo-embedded coding outputs, the patients are then placed on the paths to connect the patients to their respective communities. Moreover the patients are connected to with greater targeting accuracy to the communities that best serve the patients needs.

### CONCLUSION

All references, including granted patents and patent application publications, referred herein are incorporated herein by reference in their entirety.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Various aspects of the invention have been described above by way of illustration, and the specific embodiments disclosed are not intended to limit the invention to the particular forms disclosed. The particular implementation of the system provided thereof may vary depending upon the particular context or application. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

The corresponding structures, materials, acts, and equivalents of all means or step plus function elements in the claims below are intended to include any structure, material, or act for performing the function in combination with other claimed elements as specifically claimed.

What is claimed is:

1. A computer-implemented method comprising:
receiving data from a plurality of databases;
integrating the data into a computing system;
applying a machine learning system to identify patients from the integrated data to place in one or more communities, wherein the one or more communities include consumer-related data and social determinants of health data;
combining path projection, aggregation, and embedding to establish one or more paths to connect the patients to the one or more communities based on the consumer-related data and/or the social determinants of health data in the communities; and
training the machine learning system to identify a correct path among the one or more established paths to place the patients on to be connected to the one or more communities.

2. The method of claim 1, further comprising:
creating one or more algorithms to project the patients into the communities that include the social determinants of health data.

3. The method of claim 1, further comprising:
identifying information among physician network communities within the integrated data to further identify the one or more communities in which to place the patients.

4. The method of claim 1, further comprising:
identifying environmental information from the social determinants of health data to further identify the one or more communities in which to place the patients.

5. The method of claim 1, further comprising:
applying one or more algorithms to identify an economic status of at least one of the identified patients.

6. The method of claim 1, further comprising:
de-identifying the consumer data and social determinants of health data.

7. The method of claim 1, further comprising:
applying one or more algorithms to enable the patients to interact with healthcare facilities within the communities.

8. A computer program product comprising a tangible storage medium encoded with processor-readable instructions that, when executed by one or more processors, enable the computer program product to:
receive data from a plurality of databases;
integrate the data into a computing system;
apply a machine learning system to identify patients from the integrated data to place in one or more communities, wherein the one or more communities include consumer-related data and/or social determinants of health data;
combine path projection, aggregation, and embedding to establish one or more paths to connect the patients to the one or more communities based on the consumer-related data and the social determinants of health data in the communities; and
train the machine learning system to identify a correct path among the one or more established paths to place the patients on to be connected to the communities.

9. The computer program product of claim 8, wherein data from physician networks within the communities is collected to further identify the one or more communities with which to place at least one or more of the patients.

10. The computer program product of claim 8, wherein one or more algorithms are used to place the patients in the one or more communities that include social determinants of health data.

11. The computer program product of claim 8, wherein a social or economic status of at least one or more of the patients is identified in order to determine which of the communities that the at least one or patients should be placed within.

12. The computer program product of claim 8, wherein similar attributes between the patients are identified.

13. The computer program product of claim 8, wherein the social determinants of health data include employment information within the communities.

14. The computer program product of claim 8, wherein social status among the communities is identified.

15. A system comprising:
a memory configured to store instructions;
one or more processors configured to execute the instructions to perform operations to:
receive data from a plurality of databases;
integrate the data into a computing system;
apply a machine learning system to identify patients from the integrated data to place in one or more communities, wherein the one or more communities include consumer-related data and social determinants of health data;
combine path projection, aggregation, and embedding to establish one or more paths to connect the patients to the one or more communities based on the consumer-related data and/or the social determinants of health data in the communities; and
train the machine learning system to identify a correct path among the one or more established paths to place the patients on to be connected to the communities.

16. The system of claim 15, wherein the machine learning system is trained to predict the one or more communities to place one or more of the patients.

17. The system of claim 15, wherein the social determinants of health data include environmental conditions within the one or more communities.

18. The system of claim 15, wherein a geographical topology is identified to enable the patients to be placed within the one or more of the communities.

19. The system of claim 15, wherein one or more referral networks are identified within the one or more communities.

20. The system of claim 15, wherein sociometric ties of the patients to the one or more communities are identified.
